(19) United States
(12) Patent Application Publication
(10) Pub. No.: US 2025/0375136 A1
(43) Pub. Date: Dec. 11, 2025

(54) REMOVING MAGNETOCARDIOGRAPHY NOISE USING A DEEP LEARNING NETWORK TRAINED ON SYNTHETIC MAGNETOCARDIOGRAPHY

(71) Applicant: SB Technology, Inc., Tarrytown, NY (US)

(72) Inventors: Amelia Solon, Los Angeles, CA (US); Simon Tam, Providence, RI (US); Kirstin Aschbacher, San Francisco, CA (US); Geoffrey Iwata, San Francisco, CA (US); Madelaine Liddy, Kitchener (CA)

(21) Appl. No.: 19/228,671

(22) Filed: Jun. 4, 2025

Related U.S. Application Data
(60) Provisional application No. 63/656,549, filed on Jun. 5, 2024.
(60) Provisional application No. 63/729,889, filed on Dec. 9, 2024.

(51) Int. Cl. A61B 5/243; A61B 5/00

(52) U.S. Cl. CPC A61B 5/243; A61B 5/7217; A61B 2560/0242; A61B 2562/0223; A61B 2562/043

(57) ABSTRACT

The present disclosure relates to methods, systems, and apparatus, including computer programs encoded on computer storage media, for denoising magnetic measurements. An example method includes obtaining, using a plurality of magnetometers of a magnetically unshielded device, noisy magnetic measurement data of a magnetic field at least partially caused by an organ of a subject; obtaining, using one or more inertial measurement units (IMUs) of the magnetically unshielded device, a motional measurement of the plurality of magnetometers; determining cleaned magnetic field data based at least in part on the noisy magnetic measurement data and the motional measurement data; and taking an action based at least in part on the cleaned magnetic field data.

### CROSS-REFERENCE TO RELATED APPLICATIONS

This application claims the benefit under 35 U.S.C. § 119(e) of the filing date of U.S. Patent Application No. 63/656,549, for AI-Driven Recovery of Cardiac Biomarkers from an Unshielded Bedside Magnetocardiogram, which was filed on Jun. 5, 2024, and which is incorporated here by reference in its entirety. This application also claims the benefit under 35 U.S.C. § 119(e) of the filing date of U.S. Patent Application No. 63/729,889, for Denoising Methods and Systems for Magnetic Measurement, which was filed on Dec. 9, 2024, and which is incorporated here by reference in its entirety.

### TECHNICAL FIELD

The present application relates generally to medical imaging, and more specifically to denoising methods and systems for magnetic measurements.

### BACKGROUND

Measurements of biomagnetic fields in a human subject can be used to understand potential disease states. For example, magnetoencephalography (MEG) is a non-invasive technique for investigating magnetic fields produced by human brain activity. Magnetocardiography (MCG) is a non-invasive technique for measuring magnetic fields produced by electric currents in the heart. Similar to an electrocardiogram, a magnetocardiogram has morphological features such as a QRS complex, P-waves, and T-waves.

### SUMMARY

The present application describes denoising methods and systems for magnetic measurements.

In general, one innovative aspect of the subject matter described in this present disclosure can be embodied in methods that include the actions of obtaining, using a plurality of magnetometers of a magnetically unshielded device, a magnetic measurement of one or more magnetic fields associated with an organ of a subject. The actions include obtaining, using one or more inertial measurement units (IMUs) of the magnetically unshielded device, a motional measurement of the plurality of magnetometers and/or obtaining, using a chair/bed/pillow/medical device in contact with the patient's body, movement arising from the patient's body. The actions further include determining a magnetic signal and one or more magnetic noises in the magnetic measurement based on the motional measurement. A final action resulting from the denoising may be to surface an image, video, or other representation of the cleaned data to a physician for pattern inspection and the potential derivation of adjunctive diagnostic or prognostic information.

Other embodiments of these aspects include corresponding computer systems, apparatus, and computer programs recorded on one or more computer storage devices, each configured to perform the actions of the methods. A system of one or more computers to be configured to perform particular operations or actions means that the system has installed software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination.

In some implementations, the one or more magnetic noises include a first motional noise caused by motions of the plurality of magnetometers. Determining the one or more magnetic noises includes determining the magnetic signal and the first motional noise by applying a general linear model, such as multiple linear regression or correlation analyses, to the magnetic measurement based on the motional measurement.

In some implementations, magnetic noises may be characterized in non-linear domains through frequency based analyses such as wavelet decomposition, fast fourier transform, or Lomb Scargle techniques. The resultant coefficients from such decomposition may then be mathematically subtracted or residualized out of the magnetic measurement.

In some implementations, the one or more IMUs are co-located with the plurality of magnetometers. The motional measurement includes: acceleration information along a first axis; acceleration information along a second axis perpendicular to the first axis; acceleration information along a third axis perpendicular to the first axis and the second axis; and rotational information around one or more of the first axis, the second axis, and the third axis.

In some implementations, the organ is a heart, and the motions of the plurality of magnetometers are associated with a ballisto-cardiogram (BCG) of the heart. In such settings, the movement initiated by the mechanical movement of the heart is both noise and signal. To be more specific, removing the cardiac motion-correlated magnetic field noise may inadvertently remove parts of the magnetic field signal of interest. In some embodiments, one can regress out an IMU-related noise signature. In others, one might need to take a different approach, because removing the IMU-related noise signature could also remove too much desired signal, which might, for example, make the t-wave peak smaller than it should be, thereby impacting diagnostic biomarkers. Therefore, a denoising model could assess the overlap between the noise representation and the magnetometer representation by a method such as signal coherence, and employ reweighting procedures in order to avoid subtracting out too much of the true diagnostic signal during denoising.

In some implementations, the motions of the plurality of magnetometers are associated with sensor strike motions or sensor ring-down motions. Determining ring-down frequencies may be accomplished through frequency domain analyses such as fast fourier transform and the use of a filter bank strategy to remove or reweight the power in these frequency bands.

In some implementations, the motions of the plurality of magnetometers are associated with the one or more magnetic noises including a second motional noise caused by environmental microtremors. Determining the one or more magnetic noises includes: obtaining a background magnetic measurement when the subject is absent; determining spectral noise peaks in the background magnetic measurement; and determining the second motional noise based on the spectral noise peaks.

In some implementations, the one or more magnetic noises include a first environmental noise caused by a natural ambient environment. Determining the one or more magnetic noises includes determining the first environmental noise by obtaining a background magnetic measurement when the subject is absent.

In some implementations, the one or more magnetic noises include a second environmental noise, either stationary or moving, caused by a magnetic interference. Determining the one or more magnetic noises includes: determining a simulated dipole for the magnetic interference; generating a magnetic field associated with the simulated dipole; and determining the second environmental noise by applying a forward model to the magnetic field associated with the simulated dipole based on a layout of the plurality of magnetometers. In some instances, the simulated dipole can be an electric current dipole.

In some implementations, the one or more magnetic noises include a sensor noise caused by a failure of one or more of the plurality of magnetometers. Determining the one or more magnetic noises includes determining the sensor noise by determining a bad channel in the magnetic measurement or an elevated noise floor in the magnetic measurement.

In some implementations, the actions further include generating a second magnetic measurement associated with an organ of a second subject. The second magnetic measurement is generated by a shielded measurement device or by applying a simulation method to an electrical measurement of the organ of the second subject. The actions further include generating a portion of training data by applying the one or more magnetic noises to the second magnetic measurement. The actions further include training a denoising machine learning model to reject noise by providing it with paired examples of the clean (second magnetic measurement) and noisy (noise-applied second magnetic measurement) magnetic measurements. Knowing the noisy magnetic measurement input and the clean measurement output, the machine learning model learns the noisy-to-clean transfer function.

In some implementations, the actions further include obtaining, using a second magnetically unshielded device, a third magnetic measurement of an organ of a third subject. The actions further include removing one or more magnetic noises in the third magnetic measurement by applying the denoising machine learning model to the third magnetic measurement.

The details of one or more implementations of the subject matter of the present disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example system for denoising magnetic measurements.

FIG. 2 illustrates a block diagram of an example device for measuring magnetic fields of a target organ of a human subject.

FIG. 3 illustrates an example denoising process.

FIGS. 4A(I)-4C(II) illustrate results of an example process to separate a ballisto-cardiogram (BCG) noise in a magnetic measurement.

FIG. 5 illustrates an example of motional noise caused by environmental microtremors.

FIG. 6A illustrates an example magnetic measurement obtained with presence of an environmental noise.

FIG. 6B illustrates a noise-reduced magnetic signal in the magnetic measurement of FIG. 6A.

FIG. 7 is a schematic diagram illustrating an example denoising process using denoising machine learning (ML) models.

FIGS. 8A-8B illustrate examples of noise-reduced MCG data generated by applying a denoising ML model to unshielded MCG measurements.

FIG. 9 illustrates a flow chart of an example process of determining magnetic noises.

FIG. 10 illustrates an example computing system.

Like reference numbers and designations in the various drawings indicate like elements.

### DETAILED DESCRIPTION

Magnetocardiography (MCG) is a non-invasive diagnostic technique that can detect magnetic fields generated by the heart's electrical activity, and can provide higher spatial resolution than electrocardiogramaspite its potential advantages, MCG has traditionally been confined to magnetically shielded laboratory environments, limiting its use in broader clinical applications. Recently, advances in unshielded MCG have made this technology more cost-effective and portable, allowing it to be used in real world clinical settings such as hospitals and emergency rooms. However, these environments often present complex magnetic noise sources that challenge the measurement accuracy of cardiac magnetic field signals. As a result, MCG systems deployed in such settings require robust design and advanced denoising techniques.

Denoising techniques can rely on standard signal processing techniques, including frequency filters and spatial filters. In real world environments, magnetic noise sources often overlap with a MCG signal in both frequency and spatial domains, making it difficult to separate them out using conventional filters. Spatial filters can depend on a sensor geometry of a specific medical device in use and in at least some cases need to be learned in a data-driven fashion for each individual recording, which can be a slow, computationally expensive process. Thus, reliance on frequency and spatial filters can lead to slow and unstable performance when there is inconsistency between recordings. Moreover, traditional spatial filtering approaches assume a static spatial pattern of the magnetic noise sources and the MCG signal. In unshielded environments, this assumption can be violated as noise sources can move with respect to the device sensors. Therefore, existing denoising techniques typically have reduced performance in real world settings, especially in environments with complex and non-stationary magnetic noise sources.

Some aspects of the present application describe methods and systems for separating a cleaned magnetic signal from magnetic noises and developing denoising methods that are based on machine learning (ML) approaches (such as a neural network or a deep learning network). The cleaned magnetic signal can also be referred to as cleaned magnetic data or an underlying biomagnetic signal in the present disclosure. For example, a magnetic measurement of a magnetic field associated with an organ of a subject can be obtained using magnetometers of a magnetically unshielded device. A motional measurement of the magnetometers can be obtained using inertial measurement units (IMUs) physically coupled to the magnetically unshielded device such that movement of the magnetometer results in substantially similar movement of the IMU. A magnetic signal and magnetic noise sources in the magnetic measurement can be determined based at least in part on the motional measurement. For example, a motion-correlated magnetic signal and a motion-correlated noise source can be determined based on the motional measurement and noisy magnetic measurement data of the magnetic field. In some implementations, magnetic noise sources unrelated to the movement of the magnetometers (e.g., static or moving background noise sources) can also be determined. In some implementations, the separated magnetic signal and the magnetic noise sources can be used to create training data for ML based denoising methods.

Implementations of the present application can provide one or more of the following technical advantages and/or benefits. The described techniques can provide a robust and effective solution for addressing the challenge of denoising magnetic measurements in unshielded environments. By using reference sensors such as IMUs to track motions of magnetometers, the described techniques enable accurate separation of magnetic signals and magnetic noises. These techniques can capture and profile a wide range of magnetic noise sources, thereby enabling improvements upon existing denoising techniques. For example, the techniques described in the present application facilitate development of the ML based denoising algorithms by generating more realistic training data.

Although in this application some implementations are described in the context of the MCG technique, it is understood that such description is not intended to be construed in a limiting sense. The described techniques or their modifications can be equally applicable to any other techniques that measure bio-magnetic fields in a human subject, such as magnetoencephalography (MEG) for investigating human brain activity.

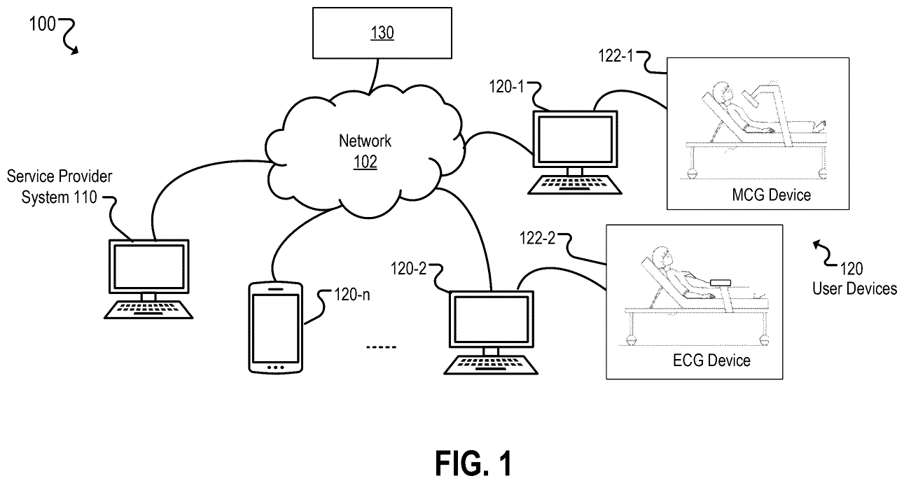

FIG. 1 illustrates an example system 100 for denoising magnetic measurements. The system 100 can include a network 102, a service provider system 110, one or more user devices 120, and a computing platform 130. The network 102 can include a large computer network, such as a local area network (LAN), a wide area network (WAN), the Internet, a cellular network, or a combination thereof, connecting any number of mobile computing devices, fixed computing devices, and server systems. The user devices 120 can be associated with one or more corresponding users. In some implementations, the user devices 120 can include user computers 120-1, 120-2, . . . , and a mobile device 120-n. The service provider system 110 can be associated with a corresponding service company or business entity. In some implementations, the service provider system 110 can communicate with users via any type of devices, systems, or servers, e.g., a desktop computer, a mobile device, a smart mobile phone, a tablet computing device, a notebook, or a portable communication device.

In some implementations, the computing platform 130 can be hosted by the user devices 120 (e.g., the user computer 120-1). In some implementations, the computing platform 130 can be hosted by the service provider system 110. In some implementations, the computing platform 130 can be hosted by multiple computers or servers connected by the network 102, and each of the multiple computers or servers can host a part of the computing platform 130. In some implementations, the system 100 can deploy a Software as a Service (SaaS) model. SaaS is a software distribution model in which a cloud provider (e.g., the computing platform 130) hosts applications and makes them available to end users (e.g., the user devices 120) over the network 102. In this model, an independent software vendor (ISV) (e.g., the service provider system 110) may contract a third-party cloud provider (e.g., Amazon web services (AWS), Microsoft Azure, Google cloud platform (GCP)) to host an application. Users can access the application through a web browser without needing to install or maintain it locally on their own computers. Examples of SaaS can include productivity tools like Google Workspace, customer relationship management (CRM) systems like Salesforce, and project management platforms like Asana. With larger companies, such as Microsoft, the cloud provider might also be a software vendor.

The system 100 can further include one or more medical devices or systems (e.g., devices 122-1 or 122-2) that are configured to measure electrical/magnetic data of an organ of a subject (e.g., a human patient). Each of the medical devices can be coupled to one of the user devices 120. The medical devices can measure the organ's electrical activity by using electrodes placed on the skin of the subject or by recording a magnetic field that the organ produces. For example, as shown in FIG. 1, the user device 120-1 can be coupled to a magnetocardiography (MCG) system 122-1. The MCG system 122-1 can include one or more magnetometers (e.g., optically pumped magnetometers (OPMs)) that are configured to measure a magnetic signal from the heart of the subject. In some implementations, the magnetic signal is a magnetic flux density. In some instances, the MCG system 122-1 (e.g., superconducting quantum interference devices (SQUID)) can be operating in a magnetically shielded room to mitigate the impact of environmental noise. In some other instances, the MCG system 122-1 can be operating in a magnetically unshielded environment. In some implementations, as shown in FIG. 1, the user device 120-2 can be coupled to an electrocardiogramaystem 122-2. The ECG system 122-2 can produce an electrocardiogram, a recording of the heart's electrical activity through repeated cardiac cycles, using electrodes placed on the subject's skin. In some other implementations (not shown in FIG. 1), the user device 120 can be coupled to a magnetoencephalography (MEG) system that measures magnetic fields produced by electrical activities of the subject's brain.

In some implementations, measured data generated by the medical devices (e.g., the MCG device 122-1 or the ECG device 122-2) coupled to the user devices 120 are transmitted (e.g., through the user device 120 and the network 102) to the computing platform 130 for further processing. The computing platform 130 can receive input data from the network 102 and generate results based on at least the input data.

The computing platform 130 can include one or more computing devices and one or more machine-readable repositories, or databases. In some implementations, the computing platform 130 can include one or more server computers in a local or distributed network each having one or more processing cores. The computing platform 130 can be implemented in a parallel processing or peer-to-peer infrastructure or on a single device with one or more processors.

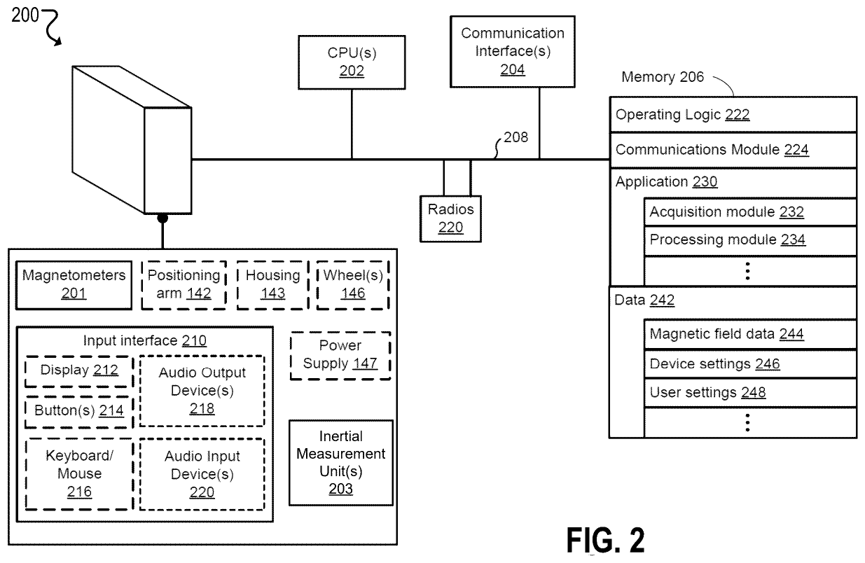

FIG. 2 shows a block diagram of an example device 200 for measuring magnetic fields of a target organ of a human subject. The device 200 can be an implementation of the MCG device 122-1 of FIG. 1. The device 200 includes one or more magnetometers 201 (e.g., a magnetic field sensor). In some implementations, the magnetometer 201 includes an electron spin defect based magnetometer, such as a diamond nitrogen vacancy (NV) center magnetometer (e.g., a solid state sensor). A diamond NV center magnetometer is a quantum sensor that leverages the occurrence of an electronic spin defect in a solid state lattice, where the spin can be both initialized and read out optically or electronically. In some instances, the defect may arise as an atomic-level vacancy in a lattice structure, such as a vacancy occurring near a nitrogen atom that is substituted in place of a carbon atom within diamond.

In some implementations, the magnetometer 201 includes an optically pumped magnetometer (OPM) (e.g., a vapor cell sensor). An OPM is a quantum sensor that includes a heated alkali vapor (including, and not limited to, a cesium vapor or a potassium vapor), through which a laser beam passes. Due to the quantum properties of the atoms, the amount of light passing through the atomic vapor is modulated at a frequency that is proportional to the environmental magnetic field.

In some implementations, the magnetometer 201 includes a scalar OPM. Scalar OPMs are highly sensitive to weak magnetic fields, have a large dynamic range (e.g., 50 μT or more), and do not require magnetic shielding or cryogenics for operation. Further, commercially available scalar OPMs are compact, lightweight, and have low power consumption, making them ideal for integration into a bedside medical device. Given the orders of magnitude separating the strength of geomagnetic fields (˜10-6 T) and cardiac fields (max ˜100 pT), scalar OPMs (because they are not vector OPMs) are sensitive only to those components of the cardiac magnetic field aligned to the total magnetic field vector, which in an unshielded environment is comprised primarily of the Earth's geomagnetic field.

In accordance with some implementations disclosed herein, the combination of excellent sensitivity, large dynamic range, high accuracy, and technical maturity make scalar OPMs especially promising for integration into a multi-channel, high-resolution MCG device. In some implementations, the scalar OPM includes a total-field OPM that can operate in the Earth's field. In some instances, the scalar OPM can be a total-field OPM.

In some implementations, the magnetometer 201 includes a fluxgate sensor.

In some implementations, the magnetometer 201 is responsive to a total magnetic field proximate to the magnetometer 201. During device operation, the magnetometer detects a total magnetic field, including bio-magnetic fields from a subject's organ as well as background magnetic field (e.g., from the earth and from other equipment in the vicinity of the total magnetometer). In some implementations, the magnetometer 201 is a scalar magnetometer that measures the total strength of the magnetic field to which it is subjected, but not the direction.

In some implementations, the magnetometer 201 is a vector magnetometer that is capable of measuring both the magnitude of the magnetic field as well as the respective field direction(s). In this case, the total strength of the magnetic field can be obtained by calibrating and processing the plurality of signals according to physical sensor orientation (e.g., computing a dot product of the vector components).

In some implementations, the use of vector magnetometers as magnetometer 201 can enable new capabilities of the device 200. Cardiac magnetic signals are directional by nature.

Using vector magnetometers enables the axial components (e.g., x-, y-, and z-components) to be fully resolved, thereby leading to higher information density and boosting source reconstruction methods. Decomposing the noise according to vector sensor axes can also improve the noise rejection algorithms. In some implementations, sensor calibration methods are provided with the vector magnetometers. For example, the vector magnetometers can be calibrated periodically, or before the use of the device 200, to ensure that orthogonality between sensors is maintained and high performance gradiometry noise subtraction can be achieved.

In some implementations, the one or more magnetometers 201 includes at least two magnetometers that are arranged in an array. In some implementations, the array of magnetometers is arranged in a stack of planes. The array of magnetometers can be proximate to the target organ when measuring the magnetic fields of the target organ. In some implementations, a distance between the array of magnetometers and the target organ is in a range between 0.5 inches and 5 inches. For example, the distance between the array of magnetometers and the target organ can be 1 inch.

In some implementations, the device 200 includes one or more inertial measurement units (IMUs) 203. The IMUs 203 can be co-located with the magnetometers 201 and can be configured to obtain a motional measurement of the magnetometers 201. For example, the IMUs 203 can be attached to the magnetometers 201. In some other implementations, the IMUs 203 can be installed on the device 200 but not directly attached to the magnetometers 201. For instance, both the IMUs 203 and the magnetometers 201 can be installed on a sensor panel. The IMUs 203 can be configured to co-move with the magnetometers 201. The IMUs 203 can be sensors configured to measure and report specific force and/or inertial data such as acceleration (and in some implementations angular velocity). In some implementations, each of the IMUs 203 can include an accelerometer and a gyroscope. The accelerometer and the gyroscope can track an orientation, position, and motion of an object (e.g., the magnetometer 201) relative to a defined reference and provide real-time motional measurements. For example, the accelerometer can measure linear acceleration along three orthogonal axes (e.g., the x-axis, the y-axis, and the z-axis), and the gyroscope can detect rotational information (e.g., rotational velocity or angular rate) around these axes.

It is understood that the example of FIG. 2 is merely for illustration purpose and is not intended to be construed in a limiting sense. In practice, the IMUs 203 that are configured to measure inertial data of the magnetometers 201 may be in any suitable location and may not be located in the device 200. For example, the IMUs 203 may be in another device separate from the device 200. In some instances, the IMUs 203 can be at a suitable location surrounding the device 200 or the subject (e.g., the patient).

In some implementations, the device 200 includes a positioning arm 142. In some implementations, the device 200 includes a housing 143. In some implementations, the device 200 includes one or more wheels 146 for supporting the device 200 (and the positioning arm 142).

In some implementations, the device 200 is coupled to a power supply 147. In some implementations, the power supply 147 is an external power supply. In some implementations, the powers supply 147 is (or includes) a battery pack that ensures backup power for safeguarding data and/or protecting device 200 in case of a power outage. In some implementations, the battery pack facilitates movement of the device 200 between rooms without having to power down the device 200.

In some implementations, the device 200 includes an input interface 210 for facilitating user input, such as a display 212, button(s) 214, a keyboard and/or mouse 216.

In some implementations, input interface 210 includes a display device 212. In some implementations, the device 200 includes input devices such as button(s) 214, and/or a keyboard/mouse 216. Alternatively, or in addition, in some implementations, the display device 212 includes a touch-sensitive surface, in which case the display device 212 is a touch-sensitive display. In some implementations, the touch-sensitive surface is configured to detect various swipe gestures (e.g., continuous gestures in vertical and/or horizontal directions) and/or other gestures (e.g., single/double tap). In computing devices that have a touch-sensitive display, a physical keyboard is optional (e.g., a soft keyboard may be displayed when keyboard entry is needed).

The input interface 210 also includes an audio output device 218, such as speakers or an audio output connection connected to speakers, earphones, or headphones. In some implementations, the apparatus 200 includes an audio input device 220 (e.g., a microphone) to capture audio (e.g., speech from a user). In some implementations, the device 200 uses a microphone and voice recognition to supplement or replace the keyboard.

The device 200 also includes one or more processors (e.g., CPU(s)) 202, one or more communication interface(s) 204 (e.g., network interface(s)), memory 206, and one or more communication buses 208 for interconnecting these components (sometimes called a chipset).

In some implementations, the device 200 includes radio(s) 220. The radio(s) 220 enable communication with one or more communication networks and allow the device 200 to communicate with other devices, such as another component in the system 100 of FIG. 1. In some implementations, the system 202 is capable of data communication using any of a variety of custom or standard wireless protocols (e.g., IEEE 802.15.4, Wi-Fi, ZigBee, 6LoWPAN, Thread, Z-Wave, Bluetooth Smart, ISA100.5A, WirelessHART, MiWi, Ultrawide Band (UWB), and/or software defined radio (SDR)) custom or standard wired protocols (e.g., Ethernet or HomePlug), and/or any other suitable communication protocol, including communication protocols not yet developed as of the filing date of this patent application.

The memory 206 includes high-speed random-access memory, such as DRAM, SRAM, DDR RAM, or other random access solid state memory devices. In some implementations, the memory includes non-volatile memory, such as one or more magnetic disk storage devices, one or more optical disk storage devices, one or more flash memory devices, or one or more other non-volatile solid state storage devices. In some implementations, the memory 206 includes one or more storage devices remotely located from one or more processor(s) 202. The memory 206, or alternatively the non-volatile memory within the memory 206, includes a non-transitory computer-readable storage medium. In some implementations, the memory 206, or the non-transitory computer-readable storage medium of the memory 206, stores the following programs, modules, and data structures, or a subset or superset thereof: operating logic 222, a communication module 224, an application 230, and data 242.

The operating logic 222 includes procedures for handling various basic system services and for performing hardware dependent tasks.

The communication module 224 (e.g., a radio communication module) connects to and communicates with other network devices (e.g., the network 102 of FIG. 1) coupled to one or more communication networks via the communication interface(s) 204 (e.g., wired or wireless).

The application 230 acquires magnetic field data (e.g., via the magnetometers 201) and/or processes the acquired data. In some implementations, the application 230 controls one or more components of the device 200 and/or other connected devices (e.g., in accordance with the acquired data). In some implementations, the application 230 includes an acquisition module 232 and a processing module 234. The acquisition module 232 acquires magnetic data (e.g., magnetic field data) from magnetometers 201. In some implementations, the magnetic data includes time-series magnetic data. The processing module 234 processes the magnetic field data.

The data 242 for the apparatus 200 includes but is not limited to magnetic field data 244, device settings 246 for the device 200 (e.g., default options, acquisition settings, and preferred user settings), and user settings 248. In some implementations, data collected during the data acquisition process is added as data 242.

FIG. 2 is intended more as a functional description of the various features that may be present rather than as a structural schematic of the implementations described herein. In practice, and as recognized by those of ordinary skill in the art, items shown separately could be combined and some items could be separated.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 206 stores a subset of the modules and data structures identified above.

Furthermore, the memory 206 may store additional modules or data structures not described above. In some implementations, a subset of the programs, modules, and/or data stored in the memory 206 are stored on and/or executed by a server system, and/or by an external device.

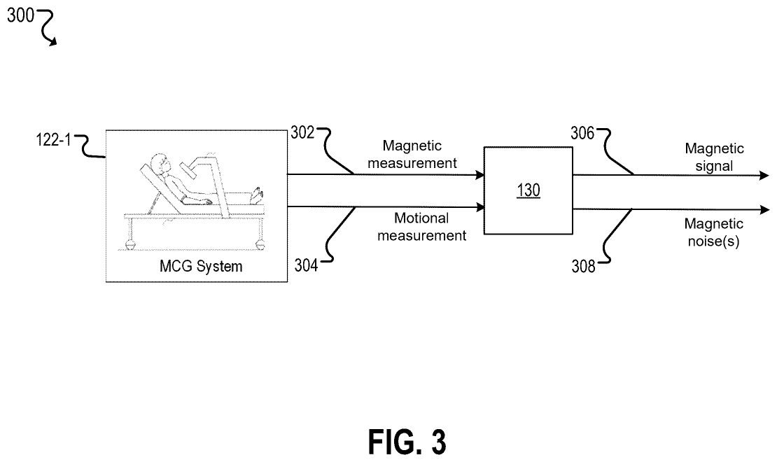

FIG. 3 illustrates an example denoising process 300. The process 300 can be performed by the computing platform 130 of FIG. 1. As shown in FIG. 3, the computing platform 130 can obtain a magnetic measurement 302 of a magnetic field associated with an organ of a subject (e.g., the heart or brain of a patient). The magnetic measurement 302 can be generated by one or more magnetometers (e.g., the magnetometers 201 of FIG. 2) of the MCG device 122-1. In some implementations, the MCG device 122-1 is an unshielded device (e.g., a magnetically unshielded device) that includes one or more IMUs (e.g., IMUs 203 as described with reference to FIG. 2). In practice, any suitable number (e.g., 2-15) of

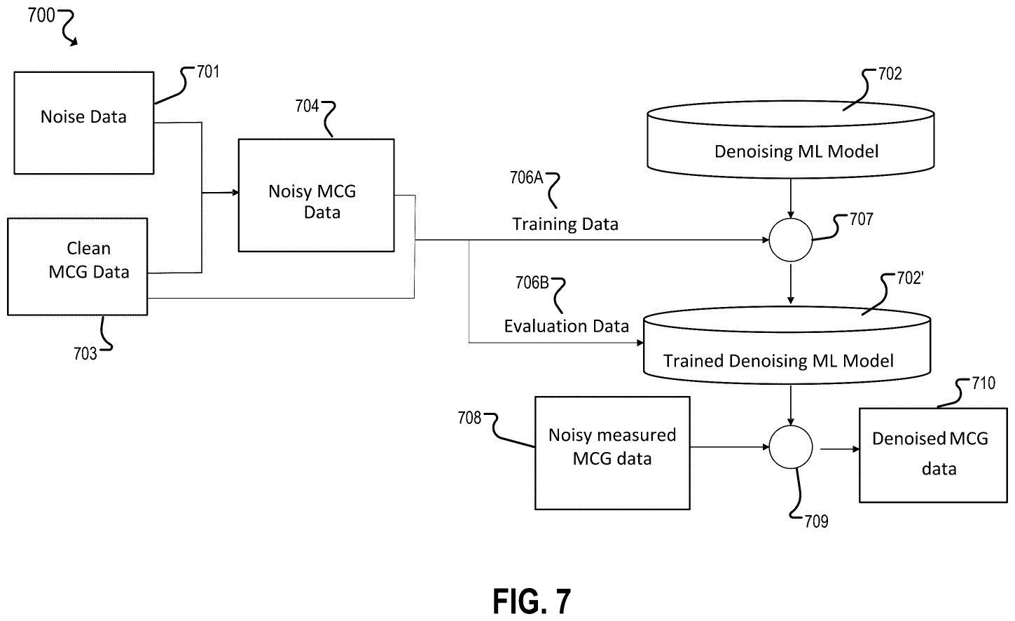

IMUs can be installed in the MCG device 122-1. The computing platform 130 can obtain a motional measurement 304 of the magnetometers of the MCG device 122-1. Data included in the motional measurement 304 can depend on capability of the IMUs 203. For example, each of the IMUs 203 can include an accelerometer, and thus the motional measurement 304 can include acceleration data along three orthogonal axes (e.g., as described with reference to FIG. 2). In some instances, the IMUs 203 are more capable and can include gyroscopes, and the motional measurement 304 can include rotational velocity around the three orthogonal axes (e.g., as described with reference to FIG. 2). The motional measurement 304 can be generated by the IMUs of the MCG device 122-1. The computing platform 130 can determine a magnetic signal 306 and one or more magnetic noise sources 308 contributing to the magnetic measurement 302 e.g., based on the motional measurement 304. In some implementations, the magnetic noise sources 308 determined in the denoising process 300 can be stored in a database as training data. The training data can be used to train a denoising ML model as described further in detail with reference to FIG. 7.

The magnetic field can be generated by the organ of the subject. In practice, the magnetic field can be affected by noise sources in an environment where the MCG device 122-1 performs the measurement. Thus, the magnetic measurement 301 can have noise components. The computing platform 130 is configured to determine one or more magnetic noise sources 308 and separate the noise source magnetic signal 306 from the overall magnetic measurement 302 to produce a cleaned magnetic field measurement of the organ. In other words, the magnetic signal 306 can be a desired magnetic signal, e.g., a noise-reduced magnetic signal representing a magnetic field produced by a specified organ of the subject.

In some implementations, the magnetic noise sources 308 can include a motional noise caused by motions of the magnetometers of the MCG device 122-1. For example, the motions of the magnetometers can be associated with a ballisto-cardiogram (BCG) of the heart. When the subject's body shakes due to the heartbeat, the magnetometers of the MCG device 122-1 (e.g., located in a sensor panel attached to the subject's body) can vibrate, and the motions of the magnetometers can cause a change to measurements of the magnetometers. The change can be referred to as a BCG artifact or a subtype of BCG noise. This subtype of the BCG noise can correlate with the motional measurement 304 generated by the IMUs of the MCG device 122-1 as the IMUs are installed to be mechanically coupled to the magnetometers of the MCG device 122-1. In other words, the IMUs can comove with the magnetometers of the MCG device 122-1. Thus, the computing platform 130 can map the motional measurement 304 from the IMUs onto the magnetometers and isolate the BCG noise that is caused by the motions of the magnetometers.

In some implementations, the magnetic signal 306 and the BCG noise can be determined by applying a multiple linear regression model to the magnetic measurement 302 based on the motional measurement 304. For example, the motional measurement 304 can include multichannel time series streams (each with x, y, and z sensing axes) from the IMUs sensors installed on the MCG device 122-1. The IMUs multichannel time series streams can be mapped to each of the magnetometers of the MCG device 122-1 based on linear regression. An example of mapping the IMU data to the MAG data is described as below:

$${B}_{0} + {\sum }_{i = 1}^{N} {B}_{i} {X}_{i} = {IMU}_{fit} , and$$

$$Y - {IMU}_{fit} = {Y}^{′} ,$$

where Y represents the magnetic measurement of a single magnetometer 302, each Xᵢ (1≤i ≤N) represents one of N IMU timeseries channels in the motional measurement 304, Bᵢ is the weight that the i-th IMU timeseries channel contributes to the linear regression fit. The error term Y′ is the remaining magnetic signal 306 because it is the residual signal that cannot fit with the IMU motion streams. Bᵢ is a coefficient/scalar. This operation can be applied to each magnetometer in the array. In some implementations, the motional measurement 304 can include only the acceleration data, or only the rotational data, or both the acceleration data and the rotational data. In some implementations, raw data output from the IMUs may include noise or other types of inaccuracy (e.g., due to sensor limitations). The motional measurement 304 can be obtained by applying suitable signal processing techniques (e.g., filtering or twice differentiation) to the raw IMU data. This approach can ensure that physically relevant inputs from the raw IMU data are used as the motional measurement 304.

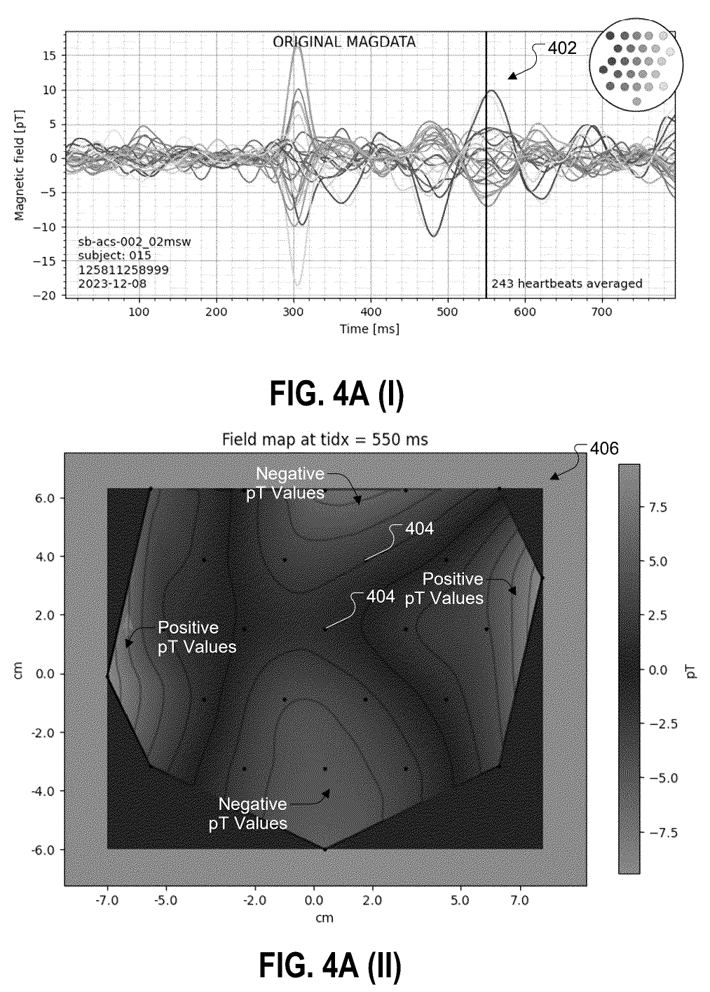

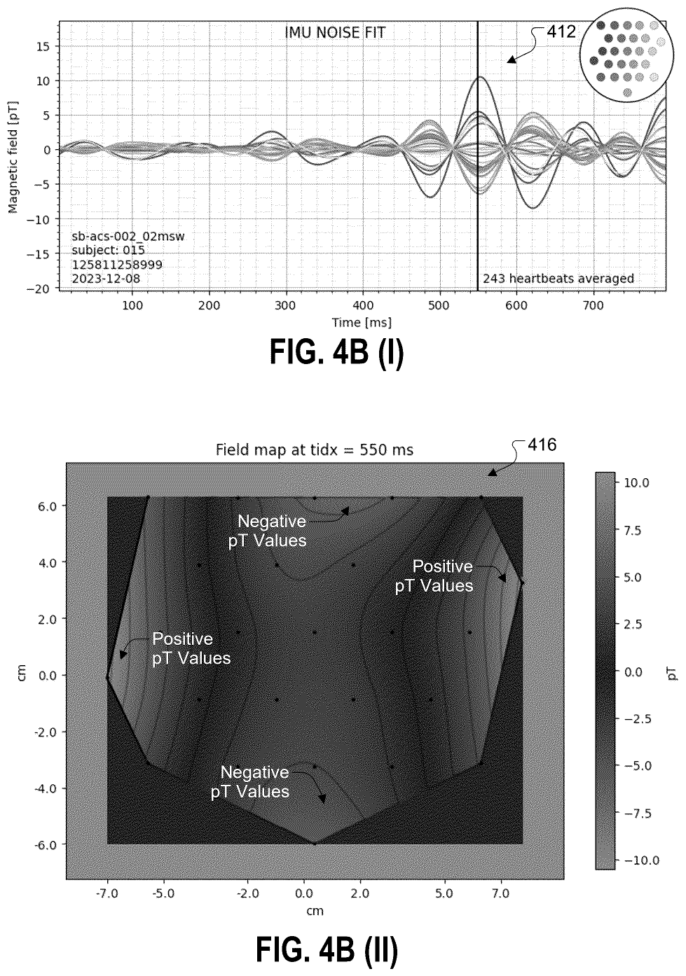

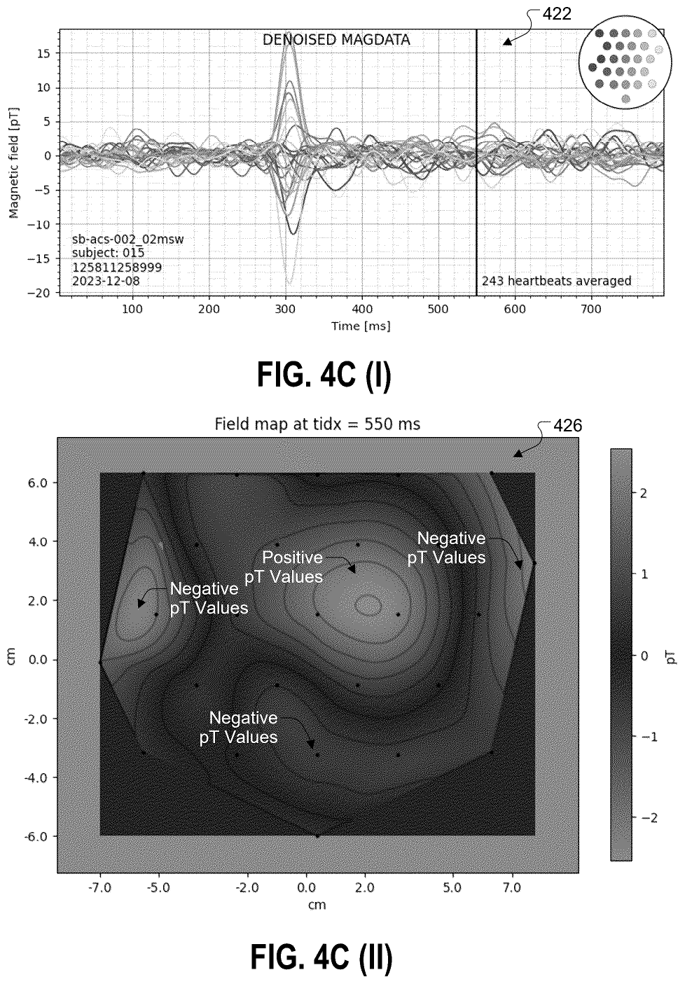

FIGS. 4A(I)-4C(II) illustrate the results of an example process to separate a BCG noise from a magnetic measurement. FIG. 4A(I) shows MCG data that includes a BCG noise. The MCG data is represented as a group of magnetic field traces 402. Each of the group of magnetic field traces 402 can be considered as magnetic field values measured by one of magnetometers 404 (as shown in FIG. 4A (II)) over the time window between 0 ms and 800 ms. The magnetometers 404 can form an array, and FIG. 4A (II) illustrates locations of the magnetometers 404 in the array relative to each other. FIG. 4A (II) also illustrates a magnetic field map (MFM) 406 at a time point 550 ms, which can be determined using interpolation methods based on the group of magnetic field traces 402 and the locations of the magnetometers 404.

The BCG noise and noise-reduced MCG data can be determined from the MCG data using the above-described method. An IMU regression method can be implemented in a sliding window (i.e. rolling buffer) approach. In a buffer keeping the last few ms or seconds of signal, one can use the input magnetic and motional measurements, do an IMU-MCG regression and BCG noise removal as described, and output the noise-reduced MCG for the given buffer.

The buffer is refreshed at a configurable time interval (or refresh rate) to provide real-time updated signals to be displayed and/or recorded. The buffer size can be adjusted to control computation load, and needs to account for noise dynamics (too short may not capture all characteristics).

Another real-time implementation can take the form of an adaptive filter where the regression coefficients are updated recursively as new magnetic and motional data is received, e.g., in discrete time steps. One can also use Huber regression or initial winsorization of outliers>abs(z-score 3) to avoid biasing regression.

FIG. 4B(I) shows a group of magnetic field traces 412 of the BCG noise, and FIG. 4B(II) shows an MFM 416 of the BCG noise at a time point 550 ms. FIG. 4C(I) shows a group of magnetic field traces 422 of the noise-reduced MCG data, and FIG. 4C(II) shows an MFM 426 of the noise-reduced MCG data at a time point 550 ms.

The BCG noise can occlude important MCG-markers, which can be used by a clinician to diagnose various heart conditions. For example, T-waves are obscured in FIGS. 4A(I) and 4A(II) due to interference of the BCG noise. In contrast, FIGS. 4C(I) and 4C(II) illustrate the T-wave (e.g., at the time point 550 ms) as the BCG noise is removed from the noise-reduced MCG data.

In some implementations, the motions of the magnetometers of the MCG device 122-1 include sensor ring-down motions. For example, when the sensors are mechanically impacted, they can oscillate at some mechanical frequency, over a settling time, until they are generally still again. The multiple linear regression model described above also can be applied to separate a motional noise caused by the sensor ring-down motions.

In some implementations, the magnetic noises 308 can include a motional noise caused by environmental microtremors. Environmental microtremors can refer to small and continuous ground vibrations that occur naturally due to various environmental factors such as human activities and natural phenomena. For example, wind blowing over the ground or through buildings can generate minor ground vibrations. In another example, the environmental microtremor can be caused by a fan in the MCG device 122-1. The environmental microtremors can cause subtle mechanical shifts in the MCG device 122-1 or the patient's position, leading to misalignment or inaccuracies in the measured magnetic fields due to the sensors moving in a background magnetic gradient. The motional noise caused by the environmental microtremors can present as spectral noise peaks observed in the magnetic measurement 302.

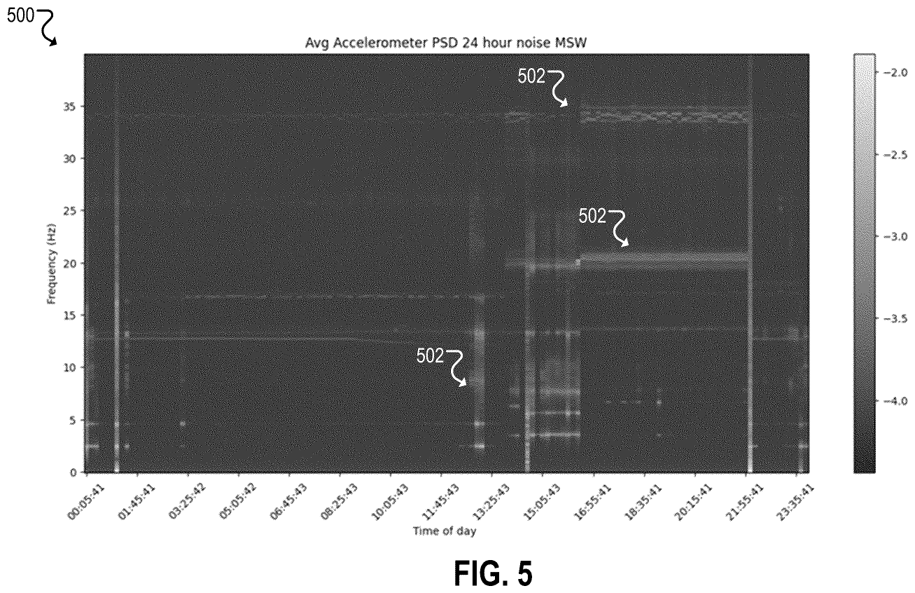

FIG. 5 illustrates an example of motional noise caused by environmental microtremors. Diagram 500 shows a magnetic measurement in the frequency domain recorded in a 24-hour time window. In the example of FIG. 5, the magnetic and inertial measurement was taken in an empty room where the patient or subject is absent. Spectral noise peaks 502 in the inertial measurement of diagram 500 represent the motional noise caused by the environmental microtremor of the device in the empty room.

In some implementations, to determine the motional noise caused by the environmental microtremors, the computing platform 130 can obtain a background magnetic and inertial measurement when the subject is absent. The computing platform 130 can determine spectral noise peaks in the background inertial measurement. The computing platform 130 can determine the motional noise based, some which may present as spectral noise peaks. For example, the computing platform 130 can separate the spectral noise peaks from the background magnetic measurement using IMU regression.

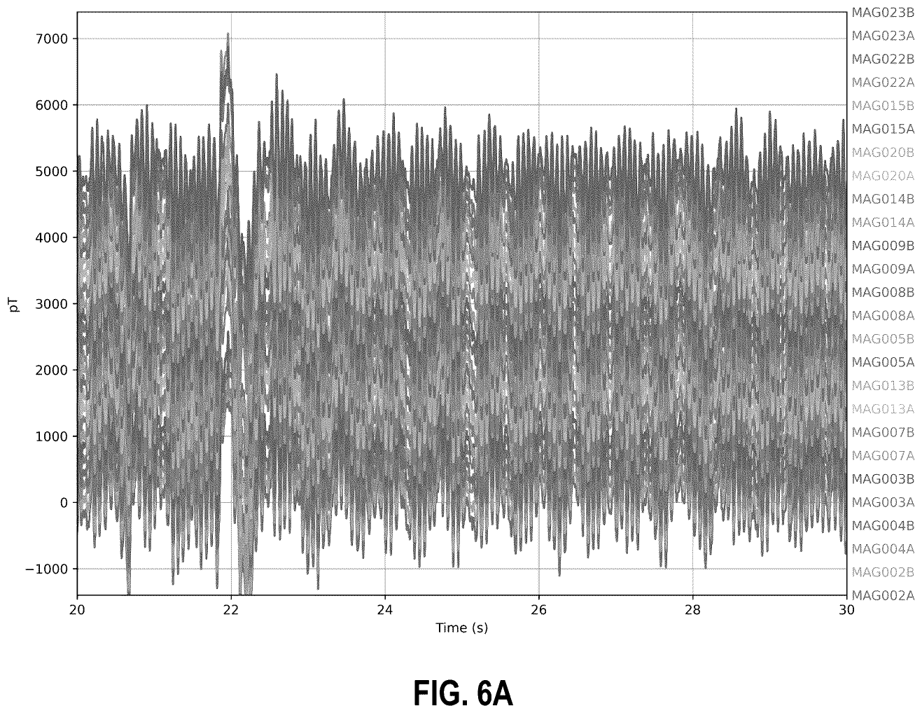

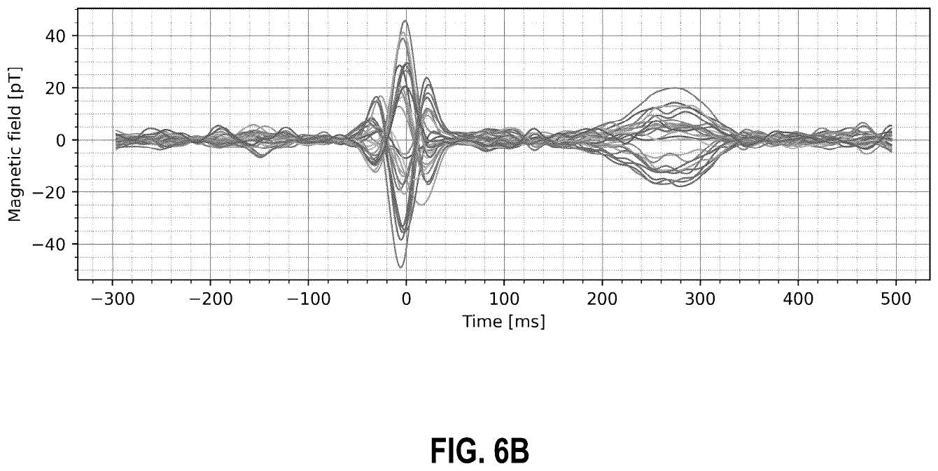

In some implementations, the magnetic noise sources 308 can include a magnetic environmental noise (also referred to as a background magnetic noise). FIG. 6A illustrates an example magnetic measurement obtained by the MCG device 122-1 with presence of an environmental magnetic noise. FIG. 6A shows a group of magnetic field traces of the magnetic measurement, and each trace is obtained by a respective magnetometer of the MCG device 122-1. FIG. 6B illustrates a noise-reduced MCG signal in the magnetic measurement of FIG. 6A. The MCG signal in FIG. 6B is approximately on the order 10 picoTesla, which is 3-5 orders of magnitude weaker than the environmental noise in the magnetic measurement. As shown in FIG. 6A, the environmental magnetic noise is a dominant component in the magnetic measurement. The environmental noise can be caused by a natural ambient environment, or an urban interference such as cars, elevators, and electronic devices, or any combination thereof. In some implementations, a magnetic implant in the subject's body such as a dental implant also contributes to the environmental noise by generating magnetic interference when the magnetic measurement 301 is obtained by the MCG device 122-1.

In some implementations, the environmental noise in the magnetic noise sources 308 that is caused by the natural ambient environment can be determined by performing magnetic measurement using “empty bed” experiments. That is, magnetic measurements can be recorded using a MCG device (e.g., the MCG device 122-1) without the presence of the subject. For example, the computing platform 130 can determine the environmental noise by obtaining a background magnetic measurement when the subject is absent or beyond a specified distance of the MCG device 122-1. In some implementations, the “empty bed” experiments can be performed in a wide range of environments each characterized by a different environment profile with varying levels of magnetic noise. Examples of the environments include a hospital environment, a non-hospital building, a laboratory, and an emergency room (which is known to be magnetically noisier). Data collected using the “empty bed” experiments can be stored in a database of magnetic noise profiles or templates.

In some implementations, the environmental noise in the magnetic noise sources 308 that is caused by a magnetic interference (cars, electronic devices, or dental implants) can be determined using a simulation-based method. The computing platform 130 can determine a simulated electric current dipole or magnetic dipole for the magnetic interference. For example, the simulated dipole data can be determined based on electrical data (e.g., a time series of ECG measurements). In some implementations, the simulated dipole can be an electric current dipole. The computing platform 130 can generate a magnetic field associated with the simulated electric current dipole, for example, by using the Biot Savart equation. In other words, the computing platform 130 can apply a forward model to the simulated electric current dipole based on a layout of the magnetometers in the MCG device 122-1 to determine the environmental noise caused by the magnetic interference. In some implementations, for example, when the magnetic interference is more complicated, the simulation-based method can simulate the magnetic interference using multiple electric current dipoles, which can be referred to as a multipole. Details of determining the magnetic field based on the simulated electric current dipole are described in U.S. Provisional Application Ser. No. 63/723,576, filed on Nov. 21, 2024. The entire disclosure of U.S. Provisional Application Ser. No. 63/723,576 is hereby incorporated by reference in its entirety.

Alternatively, in some implementations, the environmental noise in the magnetic noise sources 308 that is caused by a magnetic interference can be determined by bringing the magnetic interference close to the MCG device 122-1 and conducting the measurement. In some cases, the subject can be absent or beyond a specified distance of the MCG device 122-1. In some other cases, the magnetic interference can be attached to the subject.

FIG. 7 is a schematic diagram illustrating an example denoising process 700 using denoising ML model(s). The denoising process 700 can be implemented by a computing system (e.g., the computing platform 130 of FIG. 1) that includes one or more computing devices and one or more machine-readable repositories or databases that are in communication with each other.

The denoising process 700 can use one or more denoising techniques 702. In some implementations, the denoising techniques 702 can be referred to as signal processing. In some implementations, the denoising techniques 702 can be referred to as denoising ML model(s). In some implementations, the denoising techniques 702 can be referred to as denoising AI model(s). In some implementations, the denoising ML model(s) 702 can be referred to as artificial intelligence (AI) based denoising model(s) and can include a deep learning network (DLN) denoising model. In some implementations, the denoising ML model(s) 702 can include a denoising autoencoder. The denoising autoencoder can be a U-net, which is a convolutional neural network developed for biomedical image processing. In some implementations, the denoising autoencoder can be a diffusion-based autoencoder, which is a type of generative model that combines elements of diffusion processes with autoencoder architectures.

Clean MCG data 703 and noisy MCG data 704 can be prepared for training and/or evaluating the denoising ML models 702. In some implementations, the computing system can store the clean MCG data 703 and the noisy MCG data 704 in a repository accessible by the one or more denoising ML models 702. The clean MCG data 703 and the noisy MCG data 704 can be divided into training data 706A and evaluation data 706B.

The clean MCG data 703 may include little or no noise data. The clean MCG data 703 can be simulated MCG data. For example, the simulated MCG data can be determined by applying a simulation method to an electrical measurement (e.g., ECG data) of the heart of a subject. As described above, an example simulation method includes simulating electric activities of the organ as an electric current dipole, generating a magnetic field by applying the Biot Savart equation to the electric current dipole, and applying a forward model to the magnetic field based on a layout of the magnetometers in the MCG device 122-1. In some implementations, the clean MCG data 703 can be shielded measured MCG data. The shielded measured MCG data can be generated by the MCG system 122-1 (e.g., SQUID) in a magnetically shielded environment.

The noisy MCG data 704 (also referred to as overlaid MCG data) can be generated by adding or overlaying noise data 701 to the clean MCG data 703. In some implementations, the noise data 701 can be one or more of the magnetic noises 308 described with reference to FIG. 3. For example, the noise data 701 can be a motional noise caused by motions of the magnetometers of the MCG device 122-1, motion noise caused by motions of the subject (BCG, fidgeting, breathing), a motional noise caused by sensor strike motions or sensor ring-down motions, a motional noise caused by environmental microtremors, an environmental magnetic noise caused by a natural ambient magnetic environment, an environmental noise caused by a magnetic interference, sensor noise (e.g., caused by a malfunctioning or poorly functioning sensor) or any combination thereof. In some implementations, before being used for training and/or evaluating the denoising ML models 702, the noise data 701 can be determined (e.g., as described with reference to FIG. 3) and stored in a database by the computing system.

At a processing step 707, the computing system can train one of the denoising ML models 702 with respect to the training data 706A and return a trained denoising ML model 702′. In some implementations, after training the denoising ML model 702 with the training data 706A, the trained denoising ML model 702′ is evaluated with the evaluation data 706B. The evaluation data 706B can provide independent measure of how well the trained denoising ML model 702′ has learned to make predictions or classifications on new and/or unseen instances. In some implementations, magnetically shielded measured MCG data can be used as evaluation data 706B. During the evaluation, the magnetically shielded measured MCG data can be used as ground truth values and be compared to the predicted result (e.g., the denoised MCG data) of the trained denoising ML model 702′. In some implementations, the metric used to evaluate the performance of the trained denoising ML model 702′ is root mean square error (RMSE) between predicted results and the ground truth values. Based on the result of evaluation (e.g., RMSE), one or more parameters of the trained denoising ML model 702′ can be further adjusted.

The computing system can receive noisy measured MCG data 708 from a user device (e.g., from the user device 120 through the network 102 as illustrated in FIG. 1) and provide the noisy measured MCG data 708 as input to the trained denoising ML model 702′ running on the computing system. In the same manner as the motion regression above, a real-time implementation of the denoising ML model would use a sliding window approach. Depending on the ML model, the inputs can be either: 1. recent few seconds of magnetic measurement; or 2. Average magnetic measurements of the N recent heartbeats. Output is the cleaned magnetic measurements. In some implementations, the noisy measured MCG data 708 can be unshielded MCG data generated by the MCG system 122-1 (e.g., an unshielded MCG system) in a magnetically unshielded environment. The noisy measured MCG data 708 can be associated with the heart of a different subject.

In response, at a processing step 709, the computing system can automatically generate denoised MCG data 710 by applying the trained denoising ML 702′ to the noisy measured MCG data 708. In other words, the computing system can generate the denoised MCG data 710 by removing magnetic noises in the noisy measured MCG data 708 using the trained denoising ML model 702′. In some implementations, the trained denoising ML model 702′ can be tailored to magnetic noises in the target environment and can improve a signal to noise ratio of the noisy measured MCG data 708.

For example, a clinic can have an unshielded MCG system located in an emergency room (ER). The noise data 701 can have a magnetic noise profile according to an arrangement of the MCG system and the environment in the ER. Before the onset of patient measurement, the unshielded MCG system can be placed in its designated measurement location. The unshielded MCG system can record a background noise internally to determine the noise data 701 or at least a part of the noise data 701. The noise data 701 is then added to the clean MCG data 703 to create the noisy MCG data 704 (e.g., training and/or evaluation inputs). The denoising ML model 702 can undergo the training illustrated above in reference to FIG. 7, and the trained denoising ML model 702′ can be generated for this environment. Once the user initiates a measurement, the noisy measured MCG data 708 is inputted into the trained denoising ML model 702′. The denoised MCG data 710 can be generated for downstream use. In some implementations, if the unshielded MCG system is moved to another location, then such training can be reset and re-iterated. In some implementations, the system can reset training for new locations. In other implementations, the system can screen enough locations so that the noise data the system has accumulated becomes representative of future new locations. In other words, the model can be generalized to new locations, and be used as-is at new locations.

In some implementations, one or more parameters of the trained denoising ML model 702′ are updated based on the noisy measured MCG data 708 and the denoised MCG data 710. For example, the noisy measured MCG data 708 provided by the user device and the denoised MCG data 710 can be additionally used as the training data 706A to further train or refine the trained denoising ML model 702′, returning an updated trained denoising ML model 702′. The noisy measured MCG data 708 may contain specific patterns or anomalies relevant to their use case or environment. By incorporating this data into the training process, the trained denoising ML model 702′ can be customized to better suit the needs of users. The diversity of the training data can also be increased.

Performance of a trained denoising ML model (e.g., one of the trained denoising ML model 702′ of FIG. 7) can be evaluated in an experiment. Experimental results show that the denoising ML model can enable the recovery of predictive MCG-markers in a bedside unshielded MCG device and improve the quality of the MCG data generated by the bedside unshielded MCG device. The experiment was conducted in an urban ER to evaluate a denoising ML model trained as described with reference to FIG. 7. Specifically, the denoising ML model can be an AI based model using a deep learning network (DLN). The experiment evaluated the ability of the denoising ML model to recover empirically-supported and predictive MCG-markers of ischemia such as the T-wave magnetic field angle (T-FMA). The experiment evaluated the denoising ML model by testing that post-process MCG-markers do not significantly differ from the ground truth, unlike those from noisy synthetic (simulated) MCG data. In addition, in real-world MCG data, a simple classification algorithm (e.g., classification and regression tree (CART) model) fed MCG-markers yields better sensitivity and specificity after being processed by the denoising ML model versus after conventional signal processing (SP).

The experiment compared two data denoising methods by computing MCG-markers such as T-FMA in each condition. The first data denoising method is the denoising ML model, which is a DLN based ML model trained on synthetic or simulated MCG data and magnetic noises. The second data denoising method was a standard frequency and spatial filtering method. Both methods are applied to noisy input to reconstruct cleaned cardiac signals from the noisy input.

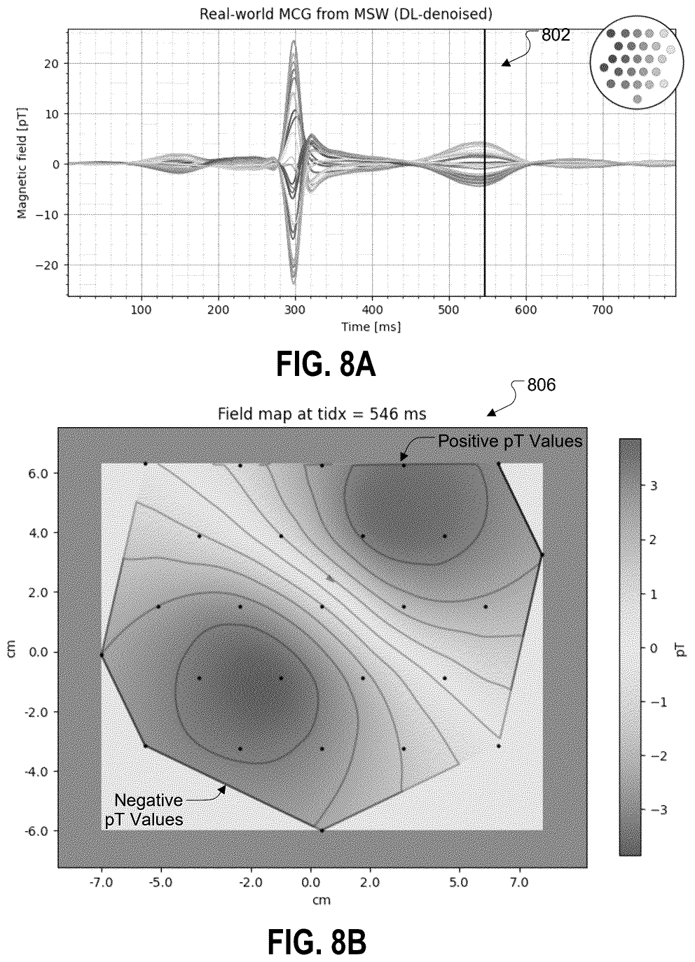

FIGS. 8A-8B illustrate example noise-reduced MCG data that is generated by applying the denoising ML model to the unshielded MCG measurements. As shown in FIG. 8A, the noise-reduced MCG data includes a group of magnetic field traces 802. Each of the group of magnetic field traces 802 can be considered as magnetic field values measured by one of magnetometers over the time window between 0 ms and 800 ms. FIG. 8B illustrates a magnetic field map 806 at a time point 546 ms, which can be determined using interpolation methods based on the group of magnetic field traces 802 and the locations of the magnetometers.

In the experiment, Wilcoxon signed-rank tests is conducted to compare denoised T-FMA means against the ground truth clean synthetic data, assuming that no difference would reflect success. To additionally validate in real-world data, the experiment used a set of 72 patients seen in an urban emergency room for suspected acute coronary syndrome. The experiment compared the diagnostic efficacy of MCG-markers of both denoising methods using a decision tree to classify these ACS rule-in (n=7) versus rule-out (n=65) patients.

The experiment results show that T-FMA did not significantly differ (p=0.228) when comparing the noise-reduced MCG data generated by the denoising ML method with the ground truth synthetic MCG data. However, the T-FMA did significantly differ (p<0.001) when comparing the noise-reduced MCG data generated by the standard frequency and spatial filtering method with the ground truth synthetic MCG data. In real-world data, the denoising ML method yielded a 15% increase in sensitivity (71% to 86%) and a 10% increase in specificity (82% to 92%), compared to the standard frequency and spatial filtering method.

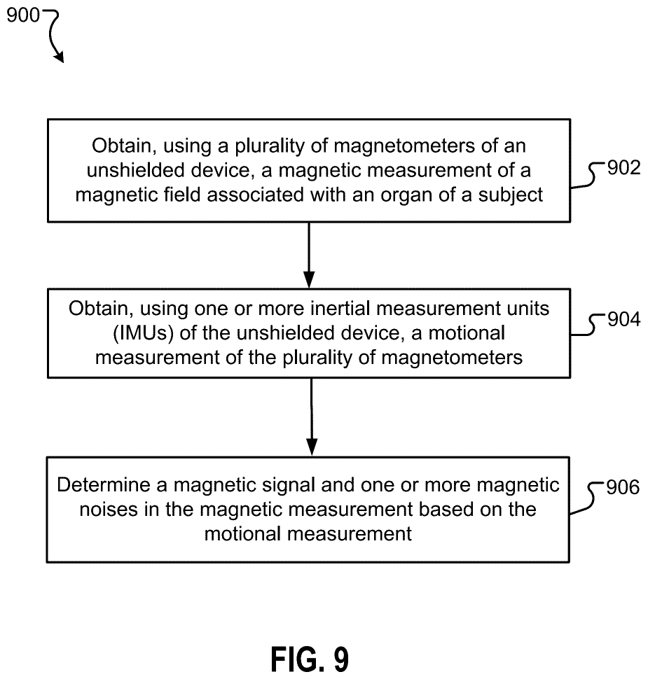

FIG. 9 illustrates a flow chart of an example process 900 of determining magnetic noises. The process 900 can be performed by any suitable computing system (e.g., the computing platform 130 of FIG. 1). It is understood that steps or operations shown in the process 900 are not exhaustive and that other operations can be performed as well before, after, or between any of the illustrated operations. Further, some of the steps or operations may be omitted, performed simultaneously or in parallel, or performed in a different order than that shown in FIG. 9.

The process 900 can begin at operation 902, where a magnetic measurement (e.g., the magnetic measurement 302 of FIG. 3) of a magnetic field associated with an organ (e.g., the heart) of a subject is obtained using a plurality of magnetometers (e.g., the magnetometers 201 of FIG. 2) of an unshielded device (e.g., the MCG device 122-1 of FIG. 1). For example, the unshielded device can be a magnetically unshielded device. In some implementations, the magnetic measurement can be noisy magnetic measurement data. At operation 904, a motional measurement (e.g., the motional measurement 304 of FIG. 3) of the plurality of magnetometers can be obtained using one or more IMUs (e.g., the IMUs 203 of FIG. 2) of the unshielded device. At operation 906, a magnetic signal (e.g., the magnetic signal 306 of FIG. 3) and one or more magnetic noises (e.g., the magnetic noises 308 of FIG. 3) in the magnetic measurement can be determined based at least in part on the motional measurement. The magnetic signal can be cleaned magnetic field data. In some implementation, determining the cleaned magnetic field data can be one of calculating, identifying, or selecting the cleaned magnetic field data based on the noisy magnetic measurement data or from the noisy magnetic measurement data.

In some implementations, the one or more magnetic noises include a first motional noise caused by motions of the plurality of magnetometers. Determining the one or more magnetic noises includes determining the magnetic signal and the first motional noise by applying a noise model to the magnetic measurement based on the motional measurement. For example, the noise model can be a multiple linear regression model. In some instances, determining the first motional noise includes isolating the first motional noise from the magnetic signal using the multiple linear regression model.

In some implementations, the one or more IMUs are co-located with the plurality of magnetometers. The motional measurement includes: acceleration information along a first axis (e.g., the x-axis); acceleration information along a second axis (e.g., the y-axis) perpendicular to the first axis; acceleration information along a third axis (e.g., the z-axis) perpendicular to the first axis and the second axis; and rotational information around one or more of the first axis, the second axis, and the third axis.

In some implementations, the organ is a heart, and the motions of the plurality of magnetometers are associated with a ballisto-cardiogram of the heart.

In some implementations, the motions of the plurality of magnetometers are associated with sensor strike motions or sensor ring-down motions.

In some implementations, the one or more magnetic noises include a second motional noise caused by environmental microtremors. Determining the one or more motional noises includes: obtaining a background magnetic and motional measurement when the subject is absent; determining spectral noise peaks in the background magnetic measurement; and determining the second motional noise based on the spectral noise peaks.

In some implementations, the one or more magnetic noises include a first environmental magnetic noise caused by a natural ambient magnetic environment. Determining the one or more magnetic noises includes determining the first environmental noise by obtaining a background magnetic measurement when the subject is absent.

In some implementations, the one or more magnetic noises include a second environmental noise caused by a magnetic interference. Determining the one or more magnetic noises includes: determining a simulated electric current dipole for the magnetic interference; generating a magnetic field associated with the simulated electric current dipole; and determining the second environmental noise by applying a forward model to the magnetic field associated with the simulated electric current dipole based on a layout of the plurality of magnetometers.

In some implementations, the process 900 further includes generating second magnetic measurement (e.g., the clean MCG data 703 of FIG. 7) associated with an organ of a second subject. The second magnetic measurement is generated by a shielded measurement device (e.g., SQUID) or by applying a simulation method to an electrical measurement of the organ of the second subject. The process 900 further includes generating a portion of training data (e.g., the training data 706A and the evaluation data 706B of FIG. 7) by applying the one or more magnetic noises to the second magnetic measurement. The process 900 further includes training a denoising ML model (e.g., the trained denoising ML model 702′ of FIG. 7) using the training data.

In some implementations, the process 900 further includes obtaining, using a second unshielded device, a third magnetic measurement (e.g., the noisy measured MCG data 708 of FIG. 7) of an organ of a third subject. The process 900 further includes removing one or more magnetic noises in the third magnetic measurement by applying the denoising ML model to the third magnetic measurement (e.g., as described in the processing step 709 of FIG. 7).

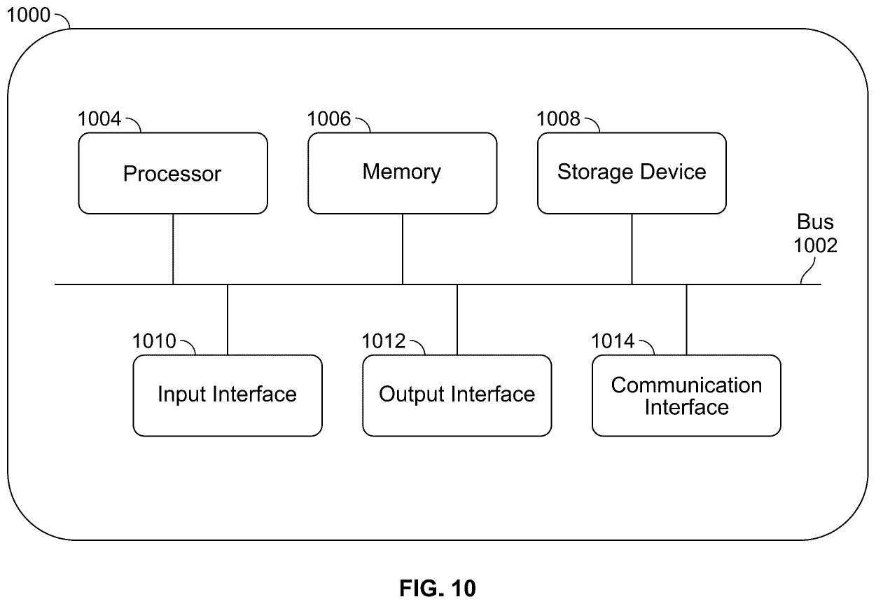

FIG. 10 illustrates an example architecture for a computing device (e.g., the service provider system 110, the user devices 120, or the computing platform 130 of FIG. 1). Referring now to FIG. 10, illustrated is a schematic diagram of a device 1000. Device 1000 includes processor 1004, memory 1006, storage component 1008, input interface 1010, output interface 1012, communication interface 1014, and bus 1002.

Bus 1002 includes a component that permits communication among the components of device 1000. In some implementations, processor 1004 is implemented in hardware, software, or a combination of hardware and software. In some examples, processor 1004 includes a processor (e.g., a central processing unit (CPU), a graphics processing unit (GPU), an accelerated processing unit (APU), and/or the like), a microphone, a digital signal processor (DSP), and/or any processing component (e.g., a field-programmable gate array (FPGA), an application specific integrated circuit (ASIC), and/or the like) that can be programmed to perform at least one function. Memory 1006 includes random access memory (RAM), read-only memory (ROM), and/or another type of dynamic and/or static storage device (e.g., flash memory, magnetic memory, optical memory, and/or the like) that stores data and/or instructions for use by processor 1004.

Storage component 1008 stores data and/or software related to the operation and use of device 1000. In some examples, storage component 1008 includes a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, a CD-ROM, RAM, PROM, EPROM, FLASH-EPROM, NV-RAM, and/or another type of computer readable medium, along with a corresponding drive.

Input interface 1010 includes a component that permits device 1000 to receive information, such as via user input (e.g., a touchscreen display, a keyboard, a keypad, a mouse, a button, a switch, a microphone, a camera, and/or the like). Additionally or alternatively, in some implementations input interface 1010 includes a sensor that senses information (e.g., a global positioning system (GPS) receiver, an accelerometer, a gyroscope, an actuator, and/or the like). Output interface 1012 includes a component that provides output information from device 1000 (e.g., a display, a speaker, one or more light-emitting diodes (LEDs), and/or the like).

In some implementations, communication interface 1014 includes a transceiver-like component (e.g., a transceiver, a separate receiver and transmitter, and/or the like) that permits device 1000 to communicate with other devices via a wired connection, a wireless connection, or a combination of wired and wireless connections. In some examples, communication interface 1014 permits device 1000 to receive information from another device and/or provide information to another device. In some examples, communication interface 1014 includes an Ethernet interface, an optical interface, a coaxial interface, an infrared interface, a radio frequency (RF) interface, a universal serial bus (USB) interface, a Wi-Fi® interface, a cellular network interface, and/or the like.

In some implementations, device 1000 performs one or more processes described herein. Device 1000 performs these processes based on processor 1004 executing software instructions stored by a computer-readable medium, such as memory 1006 and/or storage component 1008. A computer-readable medium (e.g., a non-transitory computer readable medium) is defined herein as a non-transitory memory device. A non-transitory memory device includes memory space located inside a single physical storage device or memory space spread across multiple physical storage devices.

In some implementations, software instructions are read into memory 1006 and/or storage component 1008 from another computer-readable medium or another device via communication interface 1014. When executed, software instructions stored in memory 1006 and/or storage component 1008 cause processor 1004 to perform one or more processes described herein. Additionally or alternatively, hardwired circuitry is used in place of or in combination with software instructions to perform one or more processes described herein.

Thus, implementations described herein are not limited to any specific combination of hardware circuitry and software unless explicitly stated otherwise.

Memory 1006 and/or storage component 1008 includes data storage or at least one data structure (e.g., a database and/or the like). Device 1000 is capable of receiving information from, storing information in, communicating information to, or searching information stored in the data storage or the at least one data structure in memory 1006 or storage component 1008. In some examples, the information includes network data, input data, output data, or any combination thereof.

In some implementations, device 1000 is configured to execute software instructions that are either stored in memory 1006 and/or in the memory of another device (e.g., another device that is the same as or similar to device 1000). As used herein, the term “module” refers to at least one instruction stored in memory 1006 and/or in the memory of another device that, when executed by processor 1004 and/or by a processor of another device (e.g., another device that is the same as or similar to device 1000) cause device 1000 (e.g., at least one component of device 1000) to perform one or more processes described herein. In some implementations, a module is implemented in software, firmware, hardware, and/or the like.

The number and arrangement of components illustrated in FIG. 10 are provided as an example. In some implementations, device 1000 can include additional components, fewer components, different components, or differently arranged components than those illustrated in FIG. 10. Additionally or alternatively, a set of components (e.g., one or more components) of device 1000 can perform one or more functions described as being performed by another component or another set of components of device 1000.

Embodiments of the subject matter and the functional operations described in the present disclosure can be implemented in digital electronic circuitry, in tangibly-embodied computer software or firmware, in computer hardware, including the structures disclosed in the present disclosure and their structural equivalents, or in combinations of one or more of them. Embodiments of the subject matter described in the present disclosure can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., a GPU, an FPGA (field programmable gate array), or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in the present disclosure can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated into special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in the present disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in the present disclosure can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in the present disclosure, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While the present disclosure contains many specific implementation details, these should not be construed as limitations on the scope of any invention or on the scope of what may be claimed, but rather as descriptions of features that may be specific to particular embodiments of particular inventions. Certain features that are described in the present disclosure in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method comprising:
obtaining, using a plurality of magnetometers of a magnetically unshielded device, noisy magnetic measurement data of a magnetic field at least partially caused by an organ of a subject and an ambient environment;
determining one or more sources of magnetic noise caused by the ambient environment;
determining cleaned magnetic field data associated with the organ based at least in part on the noisy magnetic measurement data and the determination of one or more sources of magnetic noise; and
taking an action based at least in part on the cleaned magnetic field data.

2. The method of claim 1, wherein the ambient environment comprises at least a stationary source of magnetic noise and a non-stationary source of magnetic noise and wherein the ambient environment comprises environmental microtremors, and wherein determining the one or more magnetic noises caused by the ambient environment comprises determining a magnetic noise caused by the environmental microtremors.

3. The method of claim 2, wherein determining the magnetic noise caused by the environmental microtremors comprises:
obtaining a background magnetic measurement when the subject is beyond a specified distance of the plurality of magnetometers;
determining spectral noise peaks in background magnetic field data; and
determining the magnetic noise caused by the environmental microtremors based on the spectral noise peaks.

4. The method of claim 1, wherein the ambient environment comprises at least a stationary source of magnetic noise and a non-stationary source of magnetic noise and wherein the ambient environment comprises a natural background magnetic field source, and wherein determining the one or more sources of magnetic noise caused by the ambient environment comprises determining magnetic noise caused by the natural background environment.

5. The method of claim 4, wherein determining the source of magnetic noise caused by the natural background environment comprises:
obtaining background magnetic field measurement data when the subject is beyond a specified distance of the plurality of magnetometers.

6. The method of claim 1, wherein the ambient environment comprises at least a stationary source of magnetic noise and a non-stationary source of magnetic noise and wherein the non-stationary source comprises an implant in the subject's body, and wherein determining the one or more sources of magnetic noise caused by the ambient environment comprises determining magnetic noise caused by the implant in the subject's body.

7. The method of claim 6, wherein determining magnetic noise caused by the implant in the subject comprises:
determining a simulated dipole for the implant;
generating a magnetic field associated with the simulated dipole by applying a forward model to the simulated dipole based on a lay out of the plurality of magnetometers; and
determining the magnetic noise caused by the implant in the subject's body based on the magnetic field.

8. The method of claim 1, wherein taking the action based at least in part on the cleaned magnetic field data comprises:
using synthetic magnetic field data and the one or more sources of magnetic noise to train a deep learning network (DLN) denoising model.

9. The method of claim 1, wherein a distance between the plurality of magnetometers and the organ is in a range between 0.5 inches and 5 inches.

10. A system comprising:
one or more computers; and
one or more storage devices on which are stored instructions that are operable, when executed by the one or more computers, to cause the one or more computers to perform a method comprising:
obtaining, using a plurality of magnetometers of a magnetically unshielded device, noisy magnetic measurement data of a magnetic field at least partially caused by an organ of a subject and an ambient environment;
determining one or more sources of magnetic noise caused by the ambient environment;
determining cleaned magnetic field data associated with the organ based at least in part on the noisy magnetic measurement data and the one or more noises; and
taking an action based at least in part on the cleaned magnetic field data.

11. The system of claim 10, wherein the ambient environment comprises at least a stationary source of magnetic noise and a non-stationary source of magnetic noise and wherein the ambient environment comprises environmental microtremors, and wherein determining the one or more sources of magnetic noise caused by the ambient environment comprises determining magnetic noise caused by the environmental microtremors.

12. The system of claim 11, wherein determining magnetic noise caused by the environmental microtremors comprises:
obtaining a background magnetic measurement when the subject is beyond a specified distance of the plurality of magnetometers;
determining spectral noise peaks in background magnetic field data; and
determining magnetic noise caused by the environmental microtremors based on the spectral noise peaks.

13. The system of claim 10, wherein the ambient environment comprises at least a stationary source of magnetic noise and a non-stationary source of magnetic noise and wherein the ambient environment comprises a natural background magnetic field source, and wherein determining the one or more sources of magnetic noises caused by the ambient environment comprises determining magnetic noise caused by sources in the natural background environment.

14. The system of claim 13, wherein determining magnetic noise caused by sources in the natural background environment comprises:
obtaining background magnetic measurement data when the subject is beyond a specified distance of the plurality of magnetometers.

15. The system of claim 10, wherein the ambient environment comprises at least a stationary source of magnetic noise and a non-stationary source of magnetic noise and wherein the non-stationary source comprises an implant in the subject's body, and wherein determining the one or more source of magnetic noise caused by the ambient environment comprises determining magnetic noise caused by the implant in the subject's body.

16. The system of claim 15, wherein determining magnetic noise caused by the implant in the subject comprises:
determining a simulated dipole for the implant;
generating an implant magnetic field associated with the simulated dipole by applying a forward model to the simulated dipole based on a lay out of the plurality of magnetometers; and
determining implant magnetic noise caused by the implant in the subject based on the implant magnetic field.

17. The system of claim 16, wherein taking the action based at least in part on the cleaned magnetic field data comprises:
using synthetic magnetic field data and the one or more sources of magnetic noise to train a deep learning network (DLN) denoising model.

18. A denoising method comprising:
measuring noisy magnetic field data proximate to an organ of a subject using an unshielded measurement device, wherein the noisy magnetic field is formed by the magnetic field produced by the organ and by sources of magnetic noise;
generating cleaned magnetic field data by applying a deep learning network (DLN) denoising model to the noisy magnetic field data, wherein the cleaned magnetic field data represents an estimation of the magnetic field data produced by the organ of the subject, the DLN denoising model having been trained using noisy training magnetic data and synthetic training magnetic data pairs; and
taking an action based on the cleaned magnetic field data.

19. The denoising method of claim 18, wherein the unshielded measurement device comprises an array of magnetometers, and a distance between the array of magnetometers and the organ is in a range between 0.5 inches and 5 inches.

20. The denoising method of claim 19, wherein taking the action based on the cleaned magnetic field data comprises one or more of:
generating a visual representation of the cleaned magnetic field data;
extracting one or more features from the cleaned magnetic field data;
training a classifier using the cleaned magnetic field data;
improving the DLN denoising model using the cleaned magnetic field data; or generating real-time operator feedback used to obtain an improved magnetic measurement.
